(19) United States
(12) Patent Application Publication
(10) Pub. No.: US 2023/0127649 A1
(43) Pub. Date: Apr. 27, 2023

(54) TRACHEOSTOMY WEANING SYSTEM AND METHOD

(71) Applicant: Quest Medical, Inc., Allen, TX (US)

(72) Inventors: Steven Parnis, Allen, TX (US); Kevin Nitzschke, Allen, TX (US); Andrew Nelson, Allen, TX (US); Jeffrey Albertsen, McKinney, TX (US); Robert Mart, Allen, TX (US); Vasu Nishtala, Frisco, TX (US)

(21) Appl. No.: 17/973,332

(22) Filed: Oct. 25, 2022

Related U.S. Application Data
(60) Provisional application No. 63/271,564, filed on Oct. 25, 2021.

(51) Int. Cl. A61M 16/04; A61M 16/20; A61M 16/00

(52) U.S. Cl. CPC A61M 16/0468; A61M 16/202; A61M 16/206; A61M 16/0051; A61M 2205/18; A61M 2205/3303

(57) ABSTRACT

An apparatus, system, and method for controlling tracheostomy weaning. The apparatus includes a lumen defining a flow path for air. The flow path is configured to communicate fluidically with an airway of a patient. A control valve coupled to the lumen is configured to automatically and selectively occlude the lumen to control a flowrate of the air passing through the lumen in real time based on respiratory data obtained from the patient.

### CROSS-REFERENCE TO RELATED APPLICATIONS

This application claims priority to U.S. Provisional Application No. 63/271,564, filed on Oct. 25, 2021, entitled “TRACHEOSTOMY WEANING SYSTEM AND METHOD.” All the applications, publications and patents listed in this paragraph are incorporated herein by reference in their entirety as examples.

### BACKGROUND

### Technical Field

Novel aspects of the present disclosure relate to the field of air flow management and more particularly to a system and method for controlling air flow through a tracheostomy tube.

### Background

Patients having trouble breathing may be provided with a breathing tube inserted at least partially into the patient's airway. Endotracheal tubes and tracheostomy tubes are two types of breathing tubes currently used to facilitate breathing. Endotracheal tubes are inserted into the mouth and at least partially into the patient's trachea. Tracheostomy tubes are inserted into a hole, i.e., a tracheostomy, in the front of the neck and directly into the trachea. Tracheostomy tubes are preferred for patients requiring long-term mechanical ventilation, often longer than two weeks.

### SUMMARY OF THE INVENTION

Novel aspects of the present disclosure are directed to a breathing apparatus comprising a lumen defining a flow path for air. The flow path is configured to communicate fluidically with an airway of a patient. A control valve coupled to the lumen automatically and selectively occludes the lumen to control a flowrate of the air passing through the lumen in real time based on respiratory data obtained from the patient.

Novel aspects of the present disclosure are also directed to a system for facilitating breathing. The system includes a breathing apparatus including a lumen defining a flow path for air. The flow path is configured to communicate fluidically with an airway of a patient. The breathing apparatus also includes a control valve coupled to the lumen. The control valve automatically and selectively occludes the lumen to control a flowrate of the air passing through the lumen in real time based on respiratory data obtained from the patient. The system also includes a set of sensors configured to capture the respiratory data from the patient, and a processor communicatively coupled to the set of sensors. The processor determines an amount oxygen in the patient's blood stream, the rate of breathing and the amount of CO2 exhaled by the patient through the lumen based on the respiratory data, and wherein the processor generates control signals for controlling the control valve to automatically control the flowrate of the air passing through the lumen in real time based on the patient's respiratory data.

Novel aspects of present disclosure are also directed to a method for facilitating breathing. The method includes the steps of determining, based on respiratory data of a patient, an amount of CO2 exhaled and oxygen required by the patient; and automatically controlling a degree of occlusion of a lumen of a breathing apparatus in real time by manipulating a control valve housed within the breathing apparatus based on the determined amount of CO2 exhaled and oxygen required.

Other aspects, embodiments and features of the invention will become apparent from the following detailed description of the invention when considered in conjunction with the accompanying figures. In the figures, each identical, or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE FIGURES

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying figures, wherein:

FIG. 1A-1B depicts conventional tracheostomy tubes for facilitating breathing;

FIG. 2 is a block diagram of a system for facilitating breathing in accordance with an illustrative embodiment;

FIG. 3 is schematic diagram of a breathing apparatus for use in a system for facilitating breathing in accordance with an illustrative embodiment;

FIGS. 4A-4C are various perspective views of a breathing apparatus in accordance with an illustrative embodiment;

FIG. 5 is a cross-sectional view of the breathing apparatus from FIG. 4A in accordance with an illustrative embodiment;

FIGS. 6A-6D are cross-sectional views of the breathing apparatus from FIG. 5 with varying degrees of occlusion in accordance with an illustrative embodiment;

FIGS. 7A-7H are schematic diagrams of lumen occlusion by another control valve in accordance with an illustrative embodiment;

FIGS. 8A-8D are schematic diagrams of lumen occlusion by another control valve in accordance with an illustrative embodiment;

FIGS. 9A-9D are schematic diagram of lumen occlusion by yet another control valve in accordance with an illustrative embodiment; and

FIG. 10 is a flowchart of a process for operating a system for facilitating breathing in accordance with an illustrative embodiment.

### DETAILED DESCRIPTION

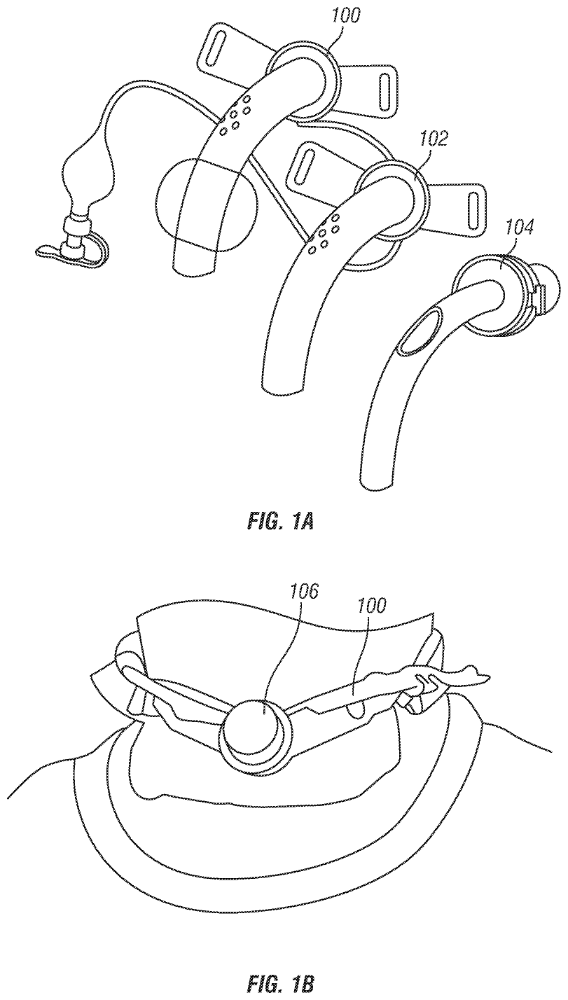

FIGS. 1A-1B depicts conventional tracheostomy tubes for facilitating breathing. In particular, FIG. 1A depicts a conventional tracheostomy tube 100 configured to be inserted into the airway of a patient, i.e., directly through a tracheostomy in a patient's neck. The process of removing the tracheostomy tube 100 is called weaning and decannulation. There are no universal guidelines or standards for weaning and decannulation. This is a methodical process which takes days to sometimes weeks to slowly wean the patient off mechanical ventilation. Sequentially smaller fenestrated (holes) cannula 102 and 104 are inserted into the tracheostomy tube 100 to gradually allow the patient to naturally take over more and more of the respiratory function. These cannulas can be replaced several times during the course of the weaning process. The final step in the weaning and decannulation process is placement of a cap 106 to cover the opening in the tracheostomy tube 100, requiring that the patient breathe without mechanical assistance, as shown in more detail in FIG. 1B.

During the weaning process, respiratory rate is manually taken, and finger oxygen saturation is the only parameter monitored. The weaning process is time-intensive, requiring application of several different downsized cannulas to progressively decrease the effective orifice. Patients often experience anxiety and may revert back to larger cannula during the weaning and decannulation process. No other automated parameters are monitored. Physicians rely on nursing personnel to monitor and provide a patient's status during the decannulation process. The process of decannulation is often slow and prolonged, which may lead to increased intensive care unit (ICU) stay, nosocomial infections, and costs. Several studies have emphasized the importance of decannulation within the ICU due to better and focused care compared to a high-dependency care unit (HDU) or ward.

Novel aspects of this disclosure provide for the following benefits: a single automated device to be used to reduce the tracheostomy orifice and replace multiple cannula; incremental and precise closure of the orifice to the tracheostomy cannula; provision of management tools to better manage and possibly accelerate the weaning process, e.g., selectable control of weaning parameters for matching individual needs; provision of various alarms, e.g., ETCO₂, respiratory rate, heart rate, and/or SpO₂; provision of smart logic to allow for programmable closure; an override system that allows a clinician to manually adjust the degree of occlusion of the lumen in real time based their professional judgement of the alarms and/or the patient's needs; integration of weaning process with EMR; simplified componentry to ensure patient contact elements are disposable and can couple with hardware system; provision of a central hardware unit and interface with multiple disposable modules to achieve multi infusion needs typically seen in ICUs; provision of a simple GUI for user to program sequential infusion delivery; multiple modalities of operation, e.g., pneumatic or electro-mechanical; and a breathing apparatus comprised of a core disposable set.

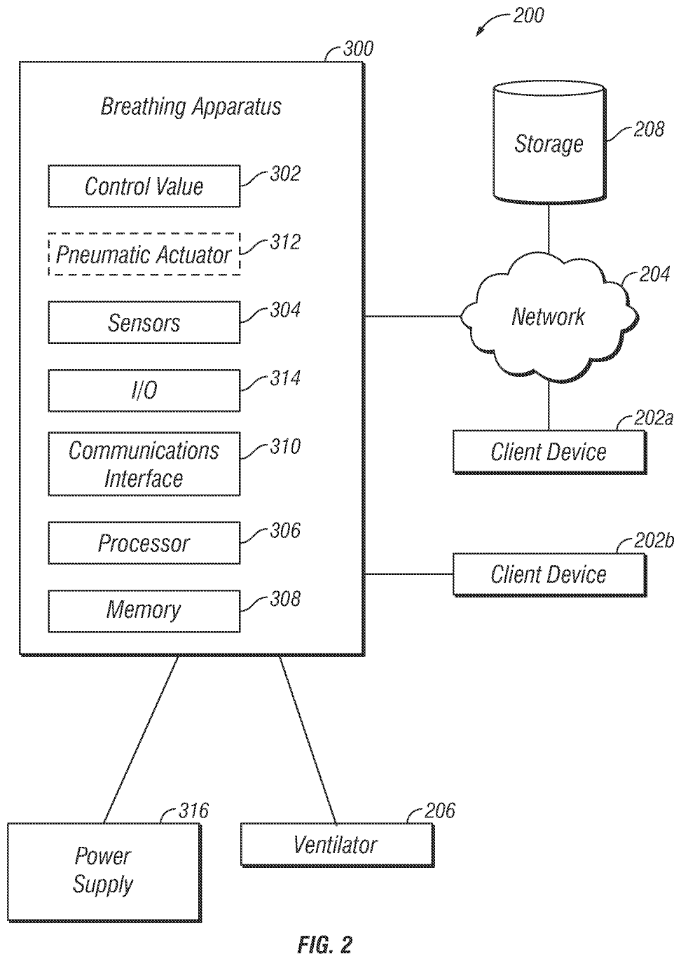

FIG. 2 is a block diagram of a system for facilitating breathing in accordance with an illustrative embodiment. The system 200 includes a breathing apparatus 300 configured manage a tracheostomy weaning process. In some embodiments, the breathing apparatus 300 can automatically control the weaning process based on respiratory data captured by the breathing apparatus 300. Embodiments of system 200 also allows a healthcare provider operating a client device 202a located remotely from the patient and the breathing apparatus 300 to monitor and manually control the weaning process, if necessary. The remote monitoring and control capability can be provided by network 204, which can be the internet. In some embodiments, the breathing apparatus 300 is connected to a local client device 202b, which allows for local monitoring and control.

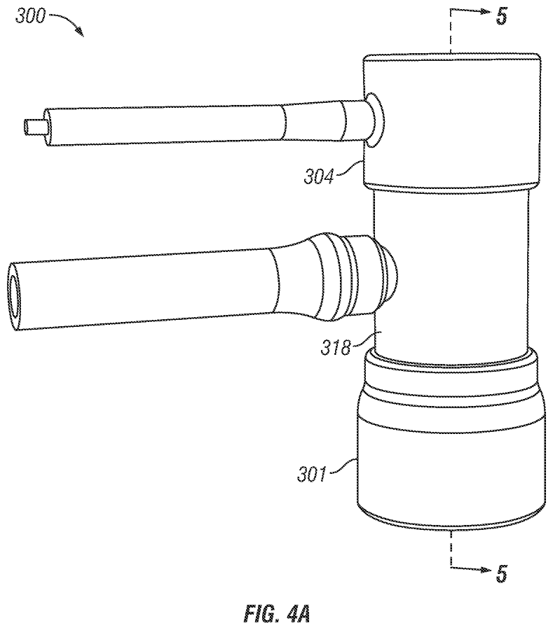

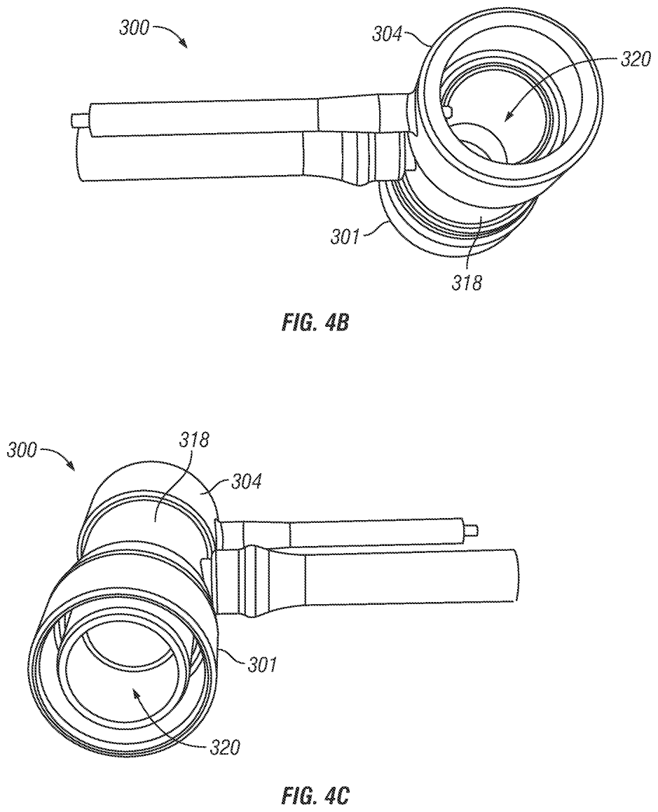

Breathing apparatus 300 includes a control valve 302 configured to selectively occlude a lumen in fluidic connection with the airway of a patient. The lumen is depicted in more detail in FIGS. 4 and 5 that follows. When a patient requires mechanical ventilation, the lumen is connected to a ventilator 206 that provides oxygen or oxygen enriched air to the patient. In the unobstructed state, the lumen provides maximum airflow. As the patient convalesces and the need for supplement oxygen and/or oxygen enriched air decreases, the control valve 302 can be controlled to gradually occlude the lumen, i.e., increase the degree of occlusion of the lumen, forcing the patient to assume greater responsibility for breathing without an abrupt change and its accompanying anxiety.

In some embodiments, the control valve 302 controls the amount of oxygen or oxygen enriched air by controlling the degree of occlusion of the lumen based on respiratory data captured by a set of sensors 304. As used herein, the term “set” means one or more. Thus, the set of sensors 304 can be a single sensor, or two or more sensors. Examples of respiratory data captured by the set of sensors 304 includes, for example and without limitation, end tidal CO2 (ETCO2), respiratory rate, pulse oximetry (sPO2), and/or heart rate.

In one embodiment, the breathing apparatus 300 includes a processor 306 for executing instructions stored in memory 308 for automatically controlling the control valve 302 based on the respiratory data. For example, the control valve 302 can reduce the degree of occlusion of the lumen passing through the breathing apparatus 300 if respiratory data indicates that the patient is not in distress and has an adequate supply of oxygen, and that the degree of occlusion of the control valve 302 has been at its current state for a predetermined period of time.

Embodiments of the breathing apparatus 300 can include a communications interface 310 for transmitting and receiving data, as in the embodiment in which a healthcare provider is remotely monitoring the weaning process from client device 202. The communications interface 310 can include currently existing or later developed wired or wireless communications technologies. The communications interface 310 can also enable the breathing apparatus 300 to integrate with the electronic medical records system to upload patient data into patient profiles. The electronic medical records system can be stored in network accessible storage devices, such as storage 208.

In some embodiments, breathing apparatus 300 can include pneumatic actuator 312 in embodiments where the control valve 302 is operated by pneumatic means. The pneumatic actuator 312 can provide air pressure for inflating and/or deflating the control valve 302 in the embodiments in which the control valve 302 includes a balloon. The pneumatic actuator 312 can include hardware for generating air pressure local to the breathing apparatus 300. Alternatively, the pneumatic actuator 312 can include hardware for selectively controlling the introduction of air pressure to the control valve 302. For example, the pneumatic actuator 312 can include an air coupler fitting that is coupled to an air conduit interfacing with the ventilator 206. The pneumatic actuator 312 can open and close the air coupler fitting to control the degree of occlusion of the lumen of the breathing apparatus 300.

In some embodiments, the breathing apparatus 300 also includes I/O 314. I/O 314 is a set of devices for providing a human-machine interface. I/O 314 can include output devices such as alarms. Alarms can be used to alert health care providers about emergent conditions of their patients. While I/O 314 is depicted as part of breathing apparatus 300, in other embodiments, I/O 314 can be alarms located remotely from the breathing apparatus 300 but triggered to sound by a wired or wireless connection via communications interface 310.

Breathing apparatus 300 can be powered by power supply 316. Power supply 316 can be an AC or DC power supply. When the power supply 316 is in the form of batteries providing DC power, the power supply 316 can be included within the housing of the breathing apparatus 300 or maintained separate from the housing of the breathing apparatus 300 and coupled together with a power cord.

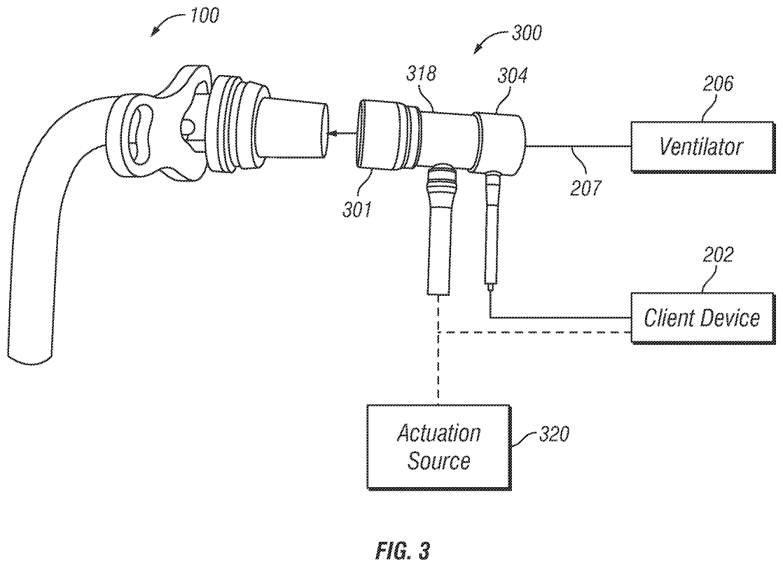

FIG. 3 is schematic diagram of a breathing apparatus for use in a system for facilitating breathing in accordance with an illustrative embodiment. In some embodiments, the breathing apparatus 300 is configured to be removably engaged with the distal end of a tracheostomy tube 100 as well as the distal end of an air conduit 207 extending from the ventilator 206.

The breathing apparatus 300 includes a housing 318 defining a lumen 320 that allows for oxygen and/or oxygen supplemented air from ventilator 206 to pass to the tracheostomy tube 100 and into the airway of the patient. As already described, a control valve 302 disposed within the housing 318 can be controlled to selectively occlude the lumen 320 to control the amount of oxygen and/or oxygen supplemented air received by the patient. In this illustrative embodiment, the control valve 302 and the corresponding hardware/software for controlling the control valve 302 are located in the middle of the breathing apparatus 300 so that the ends can be attached to one of the tracheostomy tube 100 or the air conduit 207 of the ventilator 206.

A sensor 304 is connected to an end of the breathing apparatus 300 for capturing respiratory data. In the depicted embodiment, the senor 304 is formed from a sensor housing 304a that suspends the sensing element 304b in the flow path of air passing through the breathing apparatus 300. The sensor housing 304a can be sized to receive the end of an air conduit 207 leading to the ventilator 206. In an embodiment in which the sensing element 304b is a wired sensing device, the sensing element 304b can be communicatively coupled to a client device, such as client device 202 for capturing and analyzing the captured respiratory data. In an embodiment in which the sensing element 304b is a wireless sensing device, the sensing element 304b may transmit respiratory data to a client device via the communications interface 310.

The breathing apparatus 300 depicted in FIG. 3 includes an optional adapter 301 that can secure the breathing apparatus 300 to a tracheostomy tube 100. However, in some embodiments the housing 318 of the breathing apparatus 300 can be formed to securely engage the tracheostomy tube 100 without the need for the adapter 301. For example, the housing 318 can be formed with a flared skirt configured to engage around the outer surface of the tracheostomy tube 100, or with a tapered neck configured to be inserted into the tracheostomy tube 100.

The control valve 302 of the breathing apparatus 300 can be actuated by an actuation source 320 or, in some embodiments, by the client device 202, depending upon the type of control valve 302 implemented. For example, when the control valve 302 is a pneumatic control valve, as described in more detail in FIGS. 4-6, the actuation source can be a compressor or other source of compressed air connected to the breathing apparatus 300 by an air conduit. When the control valve 302 is an electromechanical control valve, then the actuation source can be a client device, such as client device 202, connected to the breathing apparatus by a wired or wireless connection.

FIGS. 4A-4C depicts various perspective views of the breathing apparatus in accordance with an illustrative embodiment. In particular, FIG. 4A is a side view of the breathing apparatus 300, FIG. 4B is a perspective view looking into the sensor 304 of the breathing apparatus 300, and FIG. 4C is a perspective view looking into the tracheostomy tube adapter 301. The control valve 302 of breathing apparatus 300 shown in FIGS. 4A-4C is pneumatically operated and shown in more detail in the cross-section view provided in the figure that follows.

FIG. 5 is a cross-sectional view of the breathing apparatus of FIG. 4A, taken along line 5-5. The breathing apparatus 300 includes a housing 318 that defines a lumen 320. Attached to one end of the breathing apparatus 300 is a sensor housing 304a that suspends a sensing element 304b within the lumen 310. In this embodiment, the sensing element 304b is a wired sensing element, so the sensor housing 304a includes a sheath 304c that covers at least part of the wired portion of the sensing element 304b.

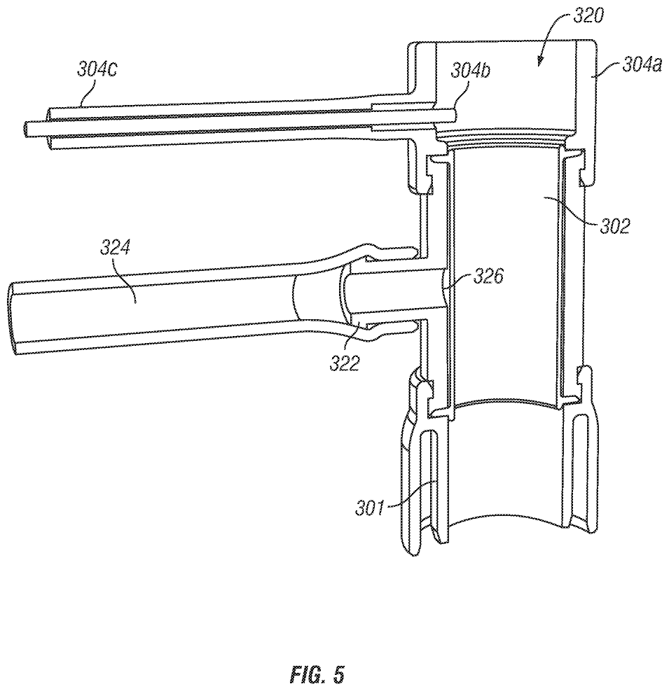

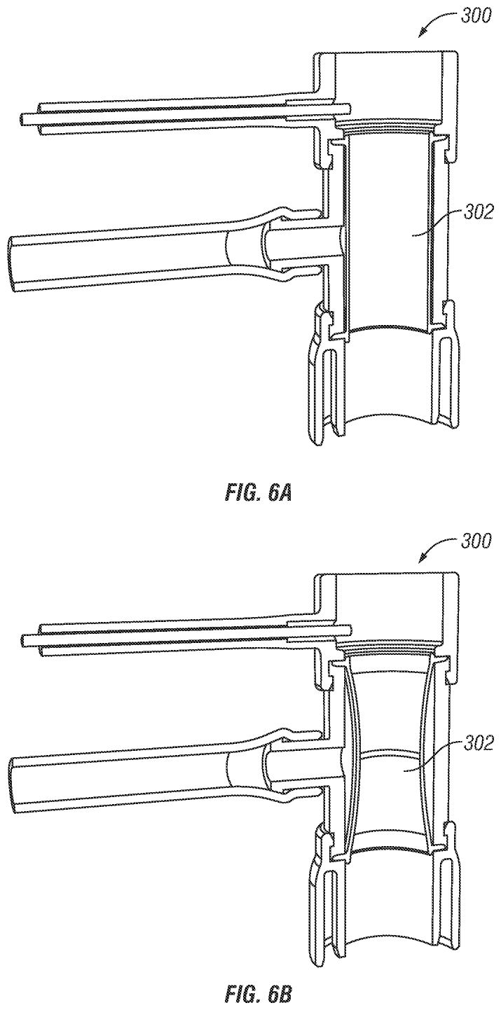

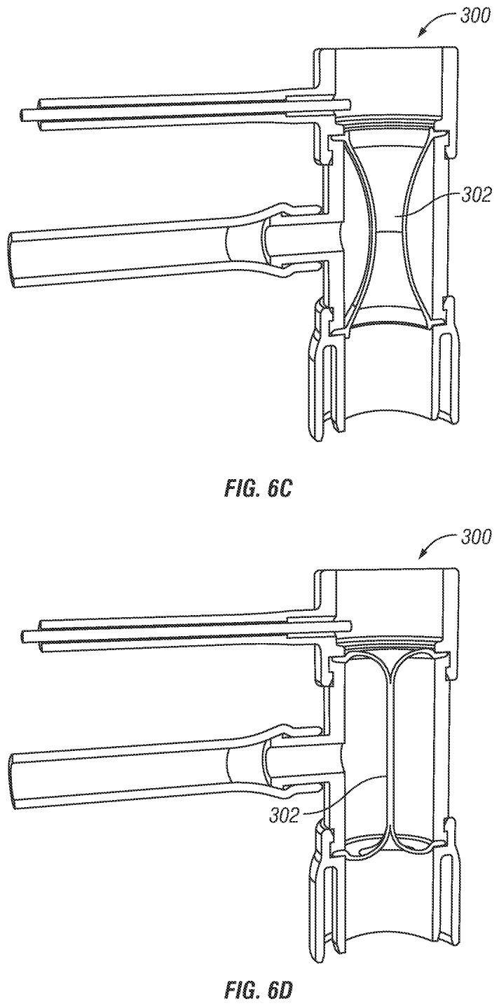

The pneumatically-operated control valve 302 in FIG. 5 is formed generally from a cylindrically-shaped membrane with its ends sealed circumferentially within the lumen 320 of the housing 318. An air coupler fitting 322 extends outwardly from the housing 318 and provides a connection point with a pneumatic line 324 connected to an air compressor that serves as an actuation source 320 in this embodiment. The air coupler fitting 322 is positioned so that its corresponding aperture 326 passing through the sidewall of the housing 318 is disposed between the sealed ends of the cylindrically-shaped membrane so that air introduced through the air coupler fitting 322 via pneumatic line 324 is introduced into the space between the cylindrically-shaped membrane and the inner surface of the housing 318. The air causes the cylindrically-shaped membrane to expand, gradually increasing a degree of occlusion until it seals the lumen 320, as shown in more detail in FIGS. 6A-6D.

FIG. 6A-6D depicts various cross-sectional views of the breathing apparatus from FIG. 5, with various increasing degrees of occlusion. In particular, FIG. 6A shows the control valve 302 without any occlusion so that air flow is completely unobstructed. FIG. 6B shows the control valve 302 with about 25% occlusion, which corresponds approximately to a 25% reduction in air flow through the lumen 320. FIG. 6C shows the control valve 302 with about 75% occlusion, which corresponds to approximately a 75% reduction in air flow through the lumen 320. FIG. 6D shows the control valve 302 with complete occlusion, which corresponds to a 100% reduction in air flow through the lumen 320.

In some embodiments, the cylindrically-shaped membrane of control valve 302 can be formed from a sheet of material having varying widths, which can provide a more predictable manner of inflation. For example, thinner membrane approximately equidistant from the ends of the cylindrically-shaped membrane can allow the central portion of the cylindrically-shaped membrane to deform more readily to increase the concavity of the cylindrically-shaped membrane during inflation. Likewise, thinner membrane at the ends of the cylindrically-shaped membrane can allow the ends of the cylindrically-shaped membrane to deform more readily to reduce the concavity of the cylindrically-shaped membrane during inflation.

The embodiment described in FIGS. 4-6 include an external source of pneumatic pressure for inflating the control valve 302. However, in another embodiment, the breathing apparatus 300 can include a hardware controller with an on-board air pressure generator controlled by a microprocessor and controlled locally or remotely via setpoint parameters provided by a medical care provider to achieve individualized management for each patient.

The setpoint parameters can be chosen to control the effective airway path size (larger or smaller airway) according to patient status and may include specific conditions set by the clinician being stable for specific time, including end tidal CO2 (ETCO2), respiratory rate, pulse oximetry (sPO2), and heart rate.

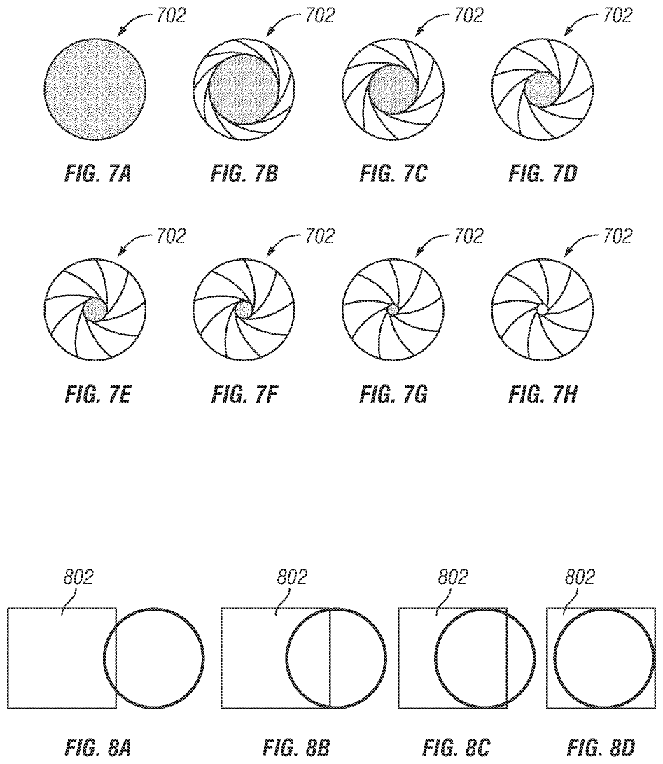

FIG. 7A-7H are schematic diagram of lumen occlusion by another control valve in accordance with an illustrative embodiment. In this embodiment an electromechanical mechanism, such as an electro-mechanical iris valve 700, is used to provide closure of the lumen. An advantage of this embodiment is more precise control of closure as compared to pneumatic actuation. This can be accomplished by movement of a mechanism to close or via a metal such as nitinol.

With reference to FIGS. 7A-7H, a control valve 702 is depicted in various states of closure. In particular, FIG. 7A depicts a completely open control valve 702, and FIGS. 7B-7H depict gradual closure of the control valve to control air flow through a lumen of a breathing apparatus. When disposed within the lumen of a breathing apparatus, such as breathing apparatus 300 in FIG. 3, the selective closure of the control valve 702 can be used to automatically control the weaning process for a patient.

FIGS. 8A-8D are schematic diagram of lumen occlusion by another control valve in accordance with an illustrative embodiment. In this embodiment, the control valve 802 is a sliding gate can be slidably positioned to selectively occlude the lumen. The control valve 802 can be actuated by pneumatic means or by electromechanical means.

With reference to FIGS. 8A-8D, the control valve 802 is shown in various states of closure. In particular, FIG. 8A depicts the control valve 802 in a substantially open configuration. FIGS. 8B and 8C depict the control valve 802 in various states of closure, and FIG. 8D depicts the control valve 802 in an entirely closed state. When properly positioned relative to a lumen of a breathing apparatus, such as breathing apparatus 300 in FIG. 3, the selective closure of the control valve 802 can be used to automatically control the weaning process for a patient.

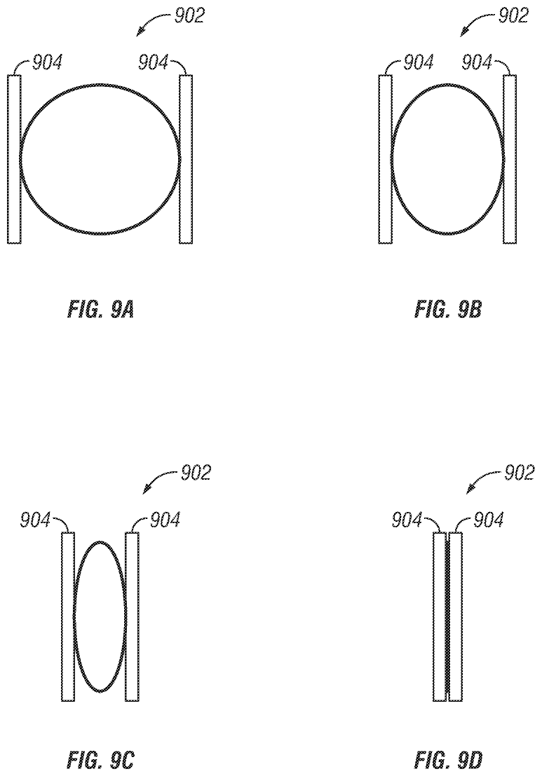

FIGS. 9A-9D are schematic diagram of lumen occlusion by yet another control valve in accordance with an illustrative embodiment. The control valve 902 can be actuated by pneumatic means or by electromechanical means.

In this embodiment, control valve 902 is a clamp engaged with an external surface of the lumen of a breathing apparatus which can be used to control a flow rate through the lumen based on a degree of closure of its clamping surfaces 904. For example, when the clamping surfaces 904 of the control valve 902 are spread furthest apart, as shown in FIG. 9A, the flowrate of air through the lumen is at a maximum. When the clamping surfaces 904 of the control valve 902 are achieve an increasingly closed state, as shown in FIGS. 9B and 9C, the flowrate of air through the lumen can be selectively reduced. In FIG. 9D, the clamping surfaces 904 of the control valve 902 have essentially sealed the lumen, preventing airflow. The selective closure of the control valve 902 can be used to automatically control the weaning process for a patient.

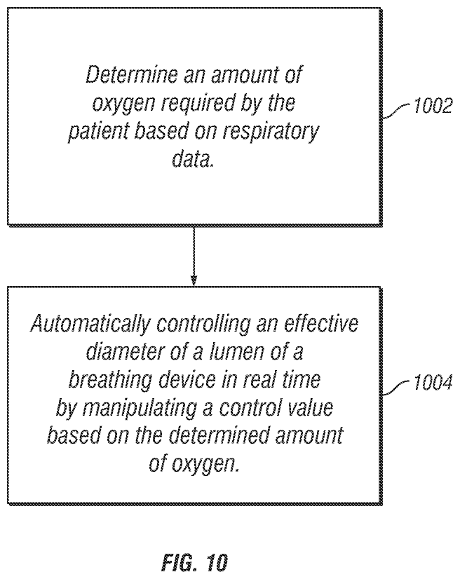

FIG. 10 is a flowchart of a process for operating a system for facilitating breathing in accordance with an illustrative embodiment. Flowchart 1000 begins at step 1002 by determining an amount of oxygen required by the patient based on respiratory data of the patient. In step 1004, the degree of occlusion of the lumen is automatically controlled in real time by manipulating a control valve housed within the breathing apparatus based on the determined amount of oxygen.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into any other element or combination of elements, or process for making or using an element or combination of elements described herein to provide additional embodiments.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. A breathing apparatus comprising:
a lumen defining a flow path for air, wherein the flow path is configured to communicate fluidically with an airway of a patient; and
a control valve coupled to the lumen, wherein the control valve automatically and selectively occludes the lumen to control a flowrate of the air passing through the lumen in real time based on respiratory data obtained from the patient.

2. The breathing apparatus of claim 1, wherein the breathing apparatus comprises a housing with a first end configured to be removably engaged with a distal end of a tracheostomy tube and a second end configured to be removably engaged with an air conduit of a ventilator.

3. The breathing apparatus of claim 1, wherein the control valve is an inflatable air bladder, and wherein the breathing apparatus further comprises:
a pneumatic actuator configured to control air pressure delivered to the inflatable air bladder.

4. The breathing apparatus of claim 3, wherein the pneumatic actuator is coupled to an air conduit of an external air supply, and wherein the pneumatic actuator controls an air coupler fitting to control the air pressure delivered to the inflatable air bladder from the external air supply.

5. The breathing apparatus of claim 1, wherein the control valve is an electromechanical valve.

6. The breathing apparatus of claim 1, wherein the breathing apparatus further comprises:
a set of sensors configured to capture the respiratory data from the patient, wherein the respiratory data is one of end tidal CO2 (ETCO2), respiratory rate, pulse oximetry (sPO2), and heart rate.

7. The breathing apparatus of claim 1, wherein breathing apparatus further comprises a communications interface configured to communicate data between the breathing apparatus and an external device.

8. The breathing apparatus of claim 7, wherein the communications interface is configured to transmit the respiratory data to the external device, and wherein the communications interface is configured to receive control data from the external device to override the automatic control of the breathing apparatus.

9. The breathing apparatus of claim 8, wherein the communications interface is configured to transmit the respiratory data to an electronic medical records database.

10. The breathing apparatus of claim 1, further comprising:
memory storing instructions;
a microprocessor configured to execute the instructions to cause the control valve to automatically and selectively occlude the lumen to control a flowrate of the air passing through the lumen in real time based on the respiratory data.

11. The breathing apparatus of claim 1, further comprising:
a set of alarms configured to notify a healthcare provider of emergent conditions of the patient based on the respiratory data.

12. The breathing apparatus of claim 1, further comprising:
a manual override system that allows a clinician to manually adjust a degree of occlusion of the lumen in real time based on one or more alarms and/or patient's needs.

13. A method for controlling breathing, the method comprising:
determining, based on respiratory data of a patient, an amount of oxygen required by the patient; and
automatically controlling a degree of occlusion of a lumen of a breathing apparatus in real time by manipulating a control valve housed within the breathing apparatus based on the determined amount of oxygen.

14. The method of claim 13, wherein determining the amount of oxygen required by the patient further comprises receiving the respiratory data captured by a set of sensors.

15. The method of claim 13, wherein automatically controlling the degree of occlusion of the lumen further comprises:
generating a control signal for controlling a pneumatic actuator configured to control air pressure delivered to an inflatable air bladder of the control valve.

16. The method of claim 15, wherein the pneumatic actuator controls an air coupler fitting connected to an external air supply, and wherein the air pressure delivered to the inflatable air bladder originates from the external air supply.

17. The method of claim 13, wherein the control valve is an electro-mechanical valve and wherein automatically controlling the degree of occlusion of the lumen further comprises:
generating a control signal for controlling operation of the electro-mechanical valve.

18. The method of claim 13, further comprising:
receiving control data from an external device to override the automatic control of the breathing apparatus.

19. The method of claim 13, further comprising:
triggering an alarm to notify a healthcare provider of emergent conditions of the patient based on the respiratory data.

20. A system comprising:
a breathing apparatus including:
a lumen defining a flow path for air, wherein the flow path is configured to communicate fluidically with an airway of a patient, and
a control valve coupled to the lumen, wherein the control valve automatically and selectively occludes the lumen to control a flowrate of the air passing through the lumen in real time based on respiratory data obtained from the patient;
a set of sensors configured to capture the respiratory data from the patient;
a processor communicatively coupled to the set of sensors, wherein the processor determines an amount of oxygen received by the patient through the lumen based on the respiratory data, and wherein the processor generates control signals for controlling the control valve to automatically control the flowrate of the air passing through the lumen in real time based on the determined amount of oxygen.
